(19) United States
(12) Patent Application Publication
(10) Pub. No.: US 2005/0027328 A1
(43) Pub. Date: Feb. 3, 2005

(54) Minimally invasive surgery placement of stimulation leads in mediastinal structures

(75) Inventor: Robert J. Greenstein, Tenafly, NJ (US)

Correspondence Address: FITCH EVEN TABIN AND FLANNERY, 120 SOUTH LA SALLE STREET, SUITE 1600, CHICAGO, IL, 60603-3406, US

(73) Assignee: Transneuronix, Inc.

(21) Appl. No.: 10/872,900

(22) Filed: Jun. 21, 2004

Related U.S. Application Data
(63) Continuation of application No. 09/961,870, filed on Sep. 24, 2001, now abandoned.
(60) Provisional application No. 60/235,659, filed on Sep. 26, 2000.

(51) Int. Cl. A61N 1/18

(52) U.S. Cl. 607/58

(57) ABSTRACT

A method for placement of electrostimulation leads in the mediastinum is provided. More particularly, this invention relates to placement of electrostimulation leads in the mediastinum using minimally invasive surgical techniques. Leads so placed may be used to stimulate specific mediastinal organs including, for example, the esophagus, neural structures such as the vagus and phrenic nerves, and cardiovascular organs such as the heart and other vessels in order to provide therapeutic, physiological, and/or patho-physiological effects on the mediastinal organs and/or target organs to which such mediastinal organs or structures are attached

### CROSS-REFERENCE TO RELATED APPLICATION

This application claims the benefit of U.S. Provisional Application No. 60/235,659, filed Sep. 26, 2000.

### FIELD OF THE INVENTION

The invention relates to placement of electrostimulation leads in the mediastinum. More particularly, this invention relates to placement of electrostimulation leads in the mediastinum using minimally invasive surgical techniques. Leads so placed may be used to stimulate specific mediastinal organs including, for example, the esophagus, neural structures such as the vagus and phrenic nerves, and cardiovascular organs such as the heart and other vessels in order to provide therapeutic, physiological, and/or patho-physiological effects on the mediastinal organs and/or target organs to which such mediastinal organs or structures are attached.

### SUMMARY OF THE INVENTION

The invention relates to placement of electrostimulation leads in the mediastinum. More particularly, this invention relates to placement of electrostimulation leads in the mediastinum using minimally invasive surgical techniques. Leads so placed may be used to stimulate specific mediastinal organs including, for example, the esophagus, neural structures such as the vagus and phrenic nerves, and cardiovascular organs such as the heart and other vessels in order to provide therapeutic, physiological, and/or patho-physiological effects on the mediastinal organs and/or target organs to which such mediastinal organs or structures are attached.

In one embodiment, suitable minimally invasive surgical instruments are used to gain access to the desired mediastinum organs by insertion from the abdominal skin under the rib cage, preferably in the area of the xyphoid. Such an approach avoids violating the pleural space. Alternatively, the desired mediastinum organs may be approached from the cervical region and through the superior mediastinum and using a celephad caudal direction.

### DESCRIPTION OF THE DRAWINGS

FIG. 1 generally illustrates the vagus nerve and the mediastinal organs innervated by the vagus nerve. The branches 10 of the vagus nerve leading to, or adjacent to, the stomach which are especially useful with electrostimulation techniques for treatment of obesity are highlighted.

FIG. 2 generally illustrates the torso with the xyphoid or xyphoid process 20 highlighted. In one embodiment of the present invention, suitable minimally invasive surgical instruments are used to gain access to the desired mediastinum organs by insertion from the abdominal skin under the rib cage, preferably in the area of the xyphoid 20 as highlighted by area 21. Such an approach avoids violating the pleural space. Alternatively, the desired mediastinum organs may be approached from the cervical region and through the superior mediastinum using a celephad caudal direction; this approach can be implemented using, for example, trocars 23.

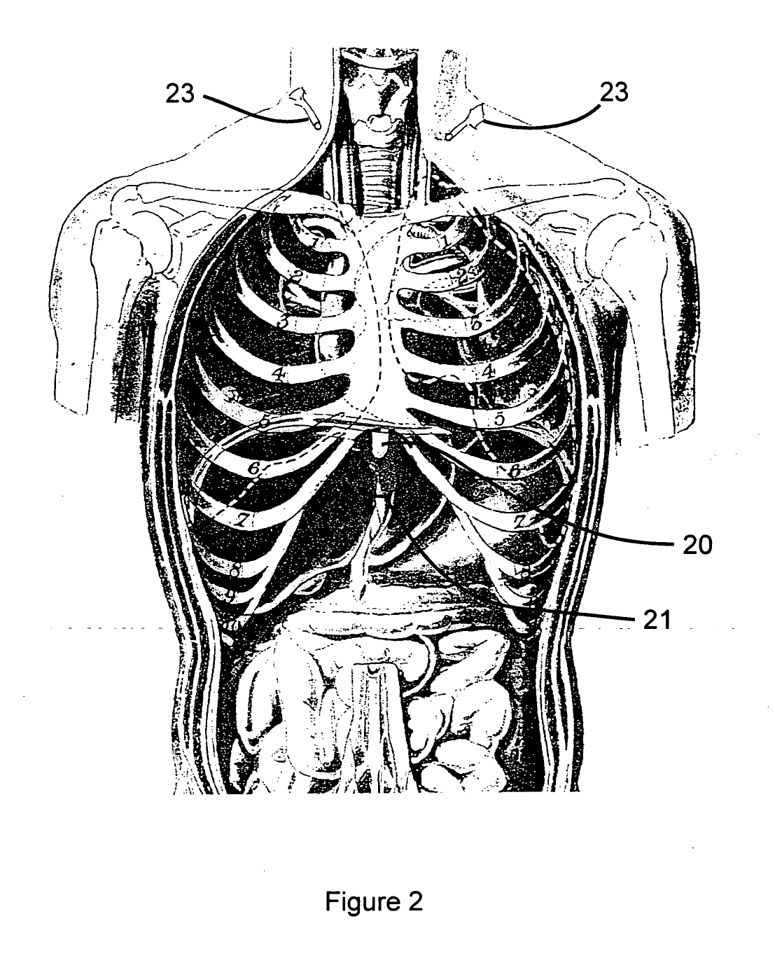

FIG. 3 also illustrates the torso with the xyphoid or xyphoid process 20 highlighted. In one embodiment of the present invention, suitable minimally invasive surgical instruments are used to gain access to the desired mediastinum organs by insertion from the abdominal skin under the rib cage as generally illustrated by arrow 22. Alternatively, the desired mediastinum organs may be approached from the cervical region and through the superior mediastinum using a celephad caudal direction as generally illustrated by arrow 24.

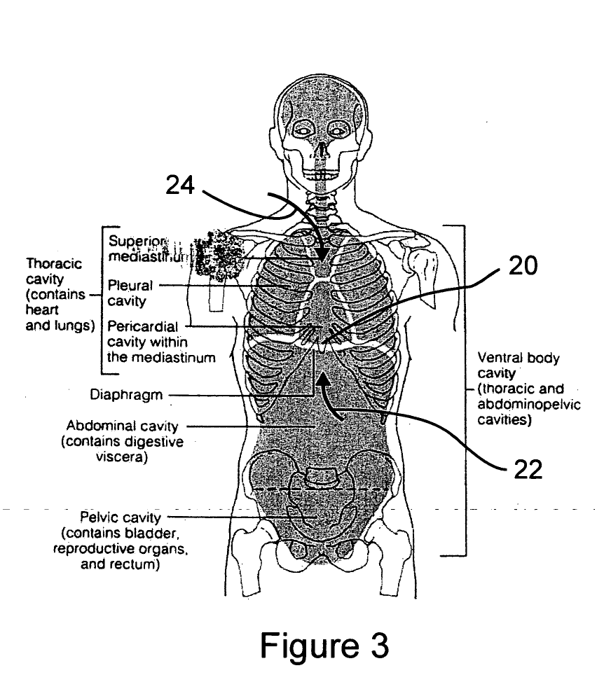

### DETAILED DESCRIPTION OF THE PREFERRED EMBODIMENTS

The invention relates to placement of electrostimulation leads in the mediastinum. More particularly, this invention relates to placement of electrostimulation leads in the mediastinum using minimally invasive surgical techniques. Leads so placed may be used to stimulate specific mediastinal organs including, for example, the esophagus, neural structures such as the vagus and phrenic nerves, and cardiovascular organs such as the heart and other vessels in order to provide therapeutic, physiological, and/or patho-physiological effects on the mediastinal organs and/or target organs to which such mediastinal organs or structures are attached.

In one embodiment, suitable minimally invasive surgical instruments are used to gain access to the desired mediastinum organs by insertion from the abdominal skin under the rib cage, preferably in the area of the xyphoid. Such an approach avoids violating the pleural space. Alternatively, the desired mediastinum organs may be approached from the cervical region and through the superior mediastinum using a celephad caudal direction.

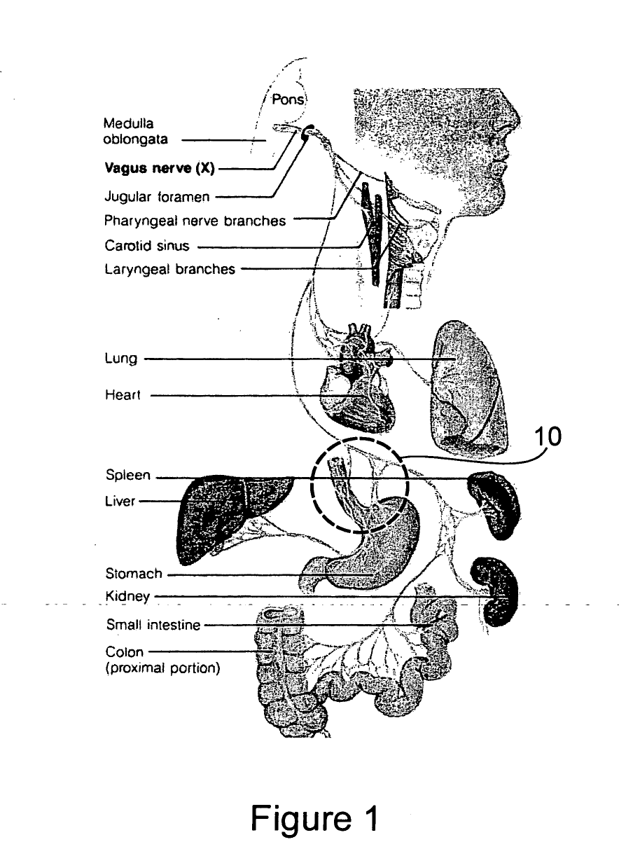

The present invention is especially adapted for placement of electrostimulation leads onto the vagus nerve and more preferably on the branches of the vagus nerve feeding the esophagus or the stomach. Placement of such electrostimulation leads on vagus nerve, or branches of the vagus nerve, leading to, or adjacent to, the stomach can be used with electrostimulation techniques for treatment of obesity. Reference 10 in FIG. 1 generally illustrates the portion of the vagus nerve especially adapted for electrostimulation for inducing weight loss in a human subject, including the control or treatment of obesity.

The present invention generally uses convention minimally invasive surgical techniques to place the desired electrostimulation device on or adjacent to the specific mediastinal organ or organs desired to be stimulated. Conventional electrostimulation devices may be used in the practice of this invention. Such devices include, for example, those described in U.S. Pat. No. 5,423,872 (Jun. 3, 1995) (an implantable gastric electrical stimulator at the antrum area of the stomach which generates sequential electrical pulses to stimulate the entire stomach, thereby artificially altering the natural gastric motility to prevent emptying or to slow down food transit through the stomach); U.S. Pat. No. 5,690,691 (Nov. 25, 1997) (a portable or implantable gastric pacemaker employing a number of electrodes along the greater curvature of the stomach for delivering phased electrical stimulation at different locations to accelerate or attenuate peristaltic movement in the GI tract); U.S. Pat. No. 5,836,994 (Nov. 17, 1998) (an implantable gastric stimulator which incorporates direct sensing of the intrinsic gastric electrical activity by one or more sensors of predetermined frequency bandwidth for application or cessation of stimulation based on the amount of sensed activity); U.S. Pat. No. 5,861,014 (Jan. 19, 1999) (an implantable gastric stimulator for sensing abnormal electrical activity of the gastrointestinal tract so as to provide electrical stimulation for a preset time period or for the duration of the abnormal electrical activity to treat gastric rhythm abnormalities); U.S. Pat. No. 6,041,258 (Mar. 21, 2000) (electrostimulation device with improved handle for laparoscopic surgery); U.S. patent application Ser. No. 09/640,201 (filed Aug. 16, 2000) (eletrostimulation device attachable to enteric or endo-abdominal tissue or viscera which is resistance to detachment); PCT Application Serial Number ______ (filed ______; Attorney Docket No. 3581/006 PCT) entitled “Gastric Stimulator Apparatus and Method for Installing” based on U.S. Provisional Application Ser. Nos. 60/129,198 and 60/129,199 (both filed Apr. 14, 1999); PCT Application Serial Number ______ (filed ______; Attorney Docket No. 3581/004 PCT) entitled “Gastric Stimulator Apparatus and Method for Use” based on U.S. Provisional Application Ser. Nos. 60/129,209 (filed Apr. 14, 1999) and 60/466,387 (filed Dec. 17, 1999); and U.S. Provisional Patent Application Serial Number ______ (filed the same date as the present application) entitled “Method and Apparatus for Intentional Impairment of Gastric Motility and/or Efficiency by Triggered Electrical Stimulation of the Gastric Tract with Respect to the Intrinsic Gastric Electrical Activity.” All of these patents, patent applications, provisional patent applications, and/or publications are hereby incorporated by reference.

1. A method for stimulating mediastinum tissue, said method comprising
(1) inserting an electrostimulation device under skin and accessing the mediastinum tissue to be stimulated, wherein the electrostimulation device has an electrostimulation lead and a distal end;
(2) attaching the electrostimulation lead to the mediastinum tissue to be stimulated;
(3) attaching the proximal end to a pulse generator; and
(3) using the pulse generator to deliver electrical stimulation through the electrostimulation lead to the mediastinum tissue to be stimulated,
wherein the insertion of the electrostimulation device and attachment of the electrostimulation lead are implemented using minimally invasive surgical techniques.

2. The method of claim 1, wherein mediastinum tissue to be treated is vagus nerve or a branch of the vagus nerve.

3. The method of claim 1, wherein mediastinum tissue to be treated is vagus nerve or a branch of the vagus nerve leading to the stomach.

4. The method of claim 1, wherein the pulse generator is an implantable and programmable pulse generator.

5. The method of claim 2, wherein the pulse generator is an implantable and programmable pulse generator.

6. The method of claim 3, wherein the pulse generator is an implantable and programmable pulse generator.

7. The method of claim 1, wherein the mediastinum tissue is accessed from abdominal skin and under rib cage.

8. The method of claim 7, wherein the abdominal skin is adjacent to xyphoid.

9. The method of claim 2, wherein the mediastinum tissue is accessed from abdominal skin and under rib cage.

10. The method of claim 9, wherein the abdominal skin is adjacent to xyphoid.

11. The method of claim 3, wherein the mediastinum tissue is accessed from abdominal skin and under rib cage.

12. The method of claim 11, wherein the abdominal skin is adjacent to xyphoid.

13. The method of claim 1, wherein the mediastinum tissue is accessed from cervical region and through superior mediastinum using a celephad caudal direction.

14. The method of claim 2, wherein the mediastinum tissue is accessed from cervical region and through superior mediastinum using a celephad caudal direction.

15. The method of claim 3, wherein the mediastinum tissue is accessed from cervical region and through superior mediastinum using a celephad caudal direction.

16. A method of inducing weight loss in a human subject, said method comprising:
(1) inserting an electrostimulation device under skin and accessing the mediastinum tissue to be stimulated, wherein the electrostimuation device has an electrostimulation lead and a distal end;
(2) attaching the electrostimulation lead to the mediastinum tissue to be stimulated;
(3) attaching the proximal end to an implantable programable pulse generator; and
(4) using the pulse generator to deliver electrical stimulation through the electrostimulation lead to the mediastinum tissue to be stimulated,
wherein the insertion of the electrostimulation device and attachment of the electrostimulation lead are implemented using minimally invasive surgical techniques.

17. The method of claim 16, wherein mediastinum tissue to be treated is vagus nerve or a branch of the vagus nerve.

18. The method of claim 16, wherein the mediastinum tissue is accessed from abdominal skin and under rib cage.

19. The method of claim 18, wherein the abdominal skin is adjacent to xyphoid.

20. The method of claim 17, wherein the mediastinum tissue is accessed from abdominal skin and under rib cage.

21. The method of claim 20, wherein the abdominal skin is adjacent to xyphoid.

22. The method of claim 16, wherein the mediastinum tissue is accessed from cervical region and through superior mediastinum using a celephad caudal direction.

23. The method of claim 17, wherein the mediastinum tissue is accessed from cervical region and through superior mediastinum using a celephad caudal direction.
